(19) United States
(12) Patent Application Publication
(10) Pub. No.: US 2016/0045472 A1
(43) Pub. Date: Feb. 18, 2016

(54) UROLITHIN B FOR MUSCLE GROWTH

(71) Applicant: PROCELL SPRL, Enghien (BE)

(72) Inventors: Fabian PRIEM, La Hulpe (BE); Julie RODRIGUEZ, Port de Bouc (FR); Marc FRANCAUX, Louvain-la-Neuve (BE); Fabienne FAUCHET, Corbais (BE)

(21) Appl. No.: 14/782,758

(22) PCT Filed: Jan. 20, 2014
(86) PCT No.: PCT/EP2014/051047

§ 371 Date: Oct. 6, 2015

(30) Foreign Application Priority Data
Jan. 18, 2013 (EP) 13151864.9

(51) Int. Cl. A61K 31/352; A61K 31/198; A61K 45/06

(52) U.S. Cl. CPC A61K 31/352; A61K 45/06; A61K 31/198

(57) ABSTRACT

A composition includes a sufficient amount of urolithin B, or a derivative thereof, for increasing muscle cell size.

### FIELD OF THE INVENTION

The present invention relates to the use of urolithin B for enhancing muscle growth and/or as androgen receptor agonist.

### BACKGROUND OF THE INVENTION AND STATE OF THE ART

Ageing is often associated with reduced muscle size and strength. Several possible mechanisms have been proposed, including mitochondrial somatic mutations, oxidative stress and low-grade inflammation. However, recent studies have touted the beneficial effect of high dose antioxidant supplementation on muscle capacities as it seems that muscle under chronic oxidative stress, but not under effort-induced oxidative stress, benefit from antioxidant supplements.

Polyphenols are well known for their antioxidant and/or anti-inflammatory properties.

One of the sources of polyphenols is pomegranate extract. During digestion, various constituants of the pomegranate extract are converted by the gut flora into urolithin C, which is then transformed into urolithin D, A, and, finally, into urolithin B. Consequently, small amounts of urolithin B can be found in the blood of human patients fed with pomegranate extract; urolithin B amount in the blood is much lower than urolithin A amount.

Beside being of lower abundance in the blood, at least in comparison with urolithin A (Gonzalez-Barrio et al., 2012), the antioxidant and anti-inflammatory properties of urolithin B are much weaker than the other related compounds, such as urolithins A, C and D, as well as a whole polyphenol extract.

Beside the antioxidant action, ellagitannin-derived compounds such as urolithin A or B, have been shown to have direct anti cancer activity, at least on breast and on prostate cancer cell lines, probably due to inhibition of testosterone-induced cell proliferation of MCF-7 cells and/or on inhibition of aromatase activity (Adams et al., 2010).

### SUMMARY OF THE INVENTION

The present invention covers a composition (preferably a food composition (thus also including a beverage), a feed composition and/or a pharmaceutical composition) comprising a sufficient amount of urolithin B and/or of a urolithin B derivative, and/or of a urolithin B precursor.

Preferably, this composition further comprises a sufficient amount of a compound (synergizing with urolithin B) selected from the group consisting of proteins (whey, egg, vegetable (soy hydrolysate, peas extracts), amino acids (e.g. leucine, valine, isoleucine, taurine), creatine, vitamins (preferably vitamin D), minerals (preferably Mg), isolated enzymes (preferably lactoferrin), polyphenols (ursolic acid, curcumin, resveratrol), capsaicin, menthol, trolamine salicylate, camphor and methylsalicylate, but also (a sufficient amount of) hydroxymethyl butyrate and/or of testosterone or of anti-myostatin (such as soluble myostatin receptor (e.g. a soluble form of activin type II receptor), blocking antibodies, siRNA). By “synergizing”, it is preferably meant that, upon addition of a sufficient amount of urolithin B (or equivalent or derivative), a lower amount of the other (synergic) compound is given than normally, while allowing the same effect (e.g. on muscle size), or that, upon addition of the other (synergic) compound at the regular dose, the presence of sufficient amount of urolithin B (or equivalent or derivative) allows an increased effect (e.g. on muscle size).

A suitable composition comprises at least 0.1% (preferably at least 0.5% or even at least 10%) of urolithin B and/or of urolithin B derivative (weight urolithin B and/or urolithin B derivative:dry weight composition), and a sufficient amount of leucine (preferably at least 5% weight, more preferably at least 10% weight, but less than 100% weight:dry weight composition).

A related (alternative) suitable composition comprises at least 10% (preferably at least 15% or even at least 50%) of urolithin B precursor (weight urolithin B precursor:dry weight composition), and a sufficient amount of leucine (preferably at least 5%, more preferably at least 10%, but less than 100% (weight:dry weight composition)).

Other related (alternative) suitable composition comprises at least 0.1% of urolithin B and/or of urolithin B derivative (w[urolithin B+urolithin B derivative]:dry weight composition), or at least 10% of urolithin B precursor (w urolithin B precursor:dry weight composition) and mixtures thereof, and a sufficient amount of leucine.

Alternatively, these compositions comprising urolithin B (and/or derivatives or precursors) comprise a sufficient amount of creatine.

Preferably, these compositions comprising urolithin B (and/or derivatives or precursors) and leucine further comprise at least 100 mg of creatine.

Preferably, these compositions (comprising urolithin B (and/or derivatives or precursors), leucine (and possibly creatine and/or other synergic compound) are edible (and/or is a beverage).

Preferably, these compositions further comprise at least an additive selected from the group consisting of vitamin D, magnesium, lactoferrin, ursolic acid and mixture thereof.

Preferably, these edible compositions (and/or beverage) have an amount of urolithin B and/or of urolithin B derivative and/or of urolithin B precursor that is sufficient for obtaining urolithin B and/or urolithin B derivative content (i.e. not urolithin A) of at least 10 nM (more preferably at least 100 nM) in the blood of a human being or of an animal (preferably a mammal, more preferably selected from the group consisting of pigs, sheep, cattle, dogs and horse), wherein preferably this mammal (human being or animal) has taken the edible composition several times (e.g. 2-4) per day or per week (e.g. 2-14) for at least 3 or even at least 6 months.

A related aspect of the present invention is a pharmaceutical composition comprising urolithin B and/or urolithin B derivative, wherein the amount of the urolithin B and/or urolithin B derivative is of at least 1 mg and/or of at least 0.1% (preferably at least 10%; weight urolithin B and/or urolithin B derivative:dry weight pharmaceutical composition) and an adequate or suitable pharmaceutical carrier or diluent.

This pharmaceutical composition can be edible or is non-edible (an injectable composition, a composition for topical administration and/or incorporated into a (dermal) patch).

Preferably, this (edible) pharmaceutical composition further comprises at least 0.25 g (preferably at least 0.5 g) of leucine and/or at least 0.1 g of creatine.

Alternatively, this (non-edible) pharmaceutical composition further comprises at least 0.01% of a compound selected from the group consisting of capsaicin, menthol, trolamine salicylate, camphor, methylsalicylate (weight compound:dry weight pharmaceutical composition). This (edible) pharmaceutical composition may also comprise (a sufficient amount of) hydroxymethyl butyrate. By sufficient amount of hydroxymethyl butyrate, it is preferably meant between 0.5 g and 5 g per day to a human adult weighting from about 60 kg to about 100 kg (e.g. a human patient affected by a myopathy or an elder human patient), more preferably between 1 g and 3 g, still more preferably between 1.5 g and 2 g. The skilled person can easily adapt (downwards, or even upwards) such dose to non-human mammal or to humans of lower weight (such as infants or severely diseased patients).

Another related aspect of the present invention is urolithin B or a derivative thereof for a therapeutic use as androgen receptor agonist.

Another related aspect of the present invention is urolithin B or a derivative thereof for use in enhancing muscle growth and/or size in (an elder) human patient or in the prevention or treatment of myopathy.

Another related aspect of the present invention is urolithin B or a derivative thereof for use in the prevention or treatment of osteoporosis in a human patient (preferably having been diagnosed as suffering or as risk of suffering of osteoporosis).

Another related aspect of the present invention is urolithin B or a derivative thereof for (a therapeutic) use in reducing muscle weight loss in a patient (e.g. an elder patient and/or a patient suffering of myopathy and/or a patient having been immobilized and/or a patient at risk of losing muscle mass).

Another related aspect of the present invention is urolithin B or a derivative thereof and a compound in synergy for muscle growth and/or repair for (a therapeutic) use in reducing muscle weight loss in a patient (e.g. an elder patient and/or a patient suffering of myopathy and/or a patient having been immobilized and/or a patient at risk of losing muscle mass).

Another related aspect of the present invention is a kit of parts comprising urolithin B, a precursor and/or a derivative thereof and another compound (preferably testosterone and/or anti-myostatin and/or hydroxymethyl butyrate) in synergy for muscle growth and/or repair.

Another related aspect of the present invention is the non-therapeutic use of urolithin B or a derivative thereof as androgen receptor agonist.

Another related aspect of the present invention is the non-therapeutical use of urolithin B or a derivative thereof for increasing muscle mass of a mammal (such as a human being or an animal selected from the group consisting of pigs, sheep, cattle, dogs, and horse or camel), or poultry or fish).

Preferably, this use of urolithin B is in synergy with another compound (such as testosterone and/or hydroxymethyl butyrate) for increasing muscle mass of a mammal.

Another related aspect of the present invention is a process to identify a non-steroid (natural) androgen receptor agonist, comprising the steps of: growing myoblast (mammalian; non human embryonic) cells, applying compositions putatively comprising an androgen agonist to these (myoblasts) cultured cells and measuring the myotube diameter of treated cells (cells were the composition is added) and of control (untreated) cells, wherein the myoblasts are grown in non-inflammatory conditions, and wherein myotubes with a bigger diameter than untreated myotubes correspond to a composition comprising at least one androgen receptor agonist.

Another related aspect of the present invention is an isolated non-steroid natural androgen-receptor agonist (being not urolithin B or a derivative thereof), possibly obtainable by the process of the present invention.

### BRIEF DESCRIPTION OF THE FIGURES

FIG. 1 represents the comparative effect of urolithin A and urolithin B on the diameter of C2Cl2 myotubes.

FIG. 2 represents the morphological results of urolithin B addition on C2Cl2 myotubes.

FIG. 3 shows that Urolithin B induces mTOR and S6K1 phosphorylation.

FIG. 4 shows that Urolithin B does not induce Akt or PRAS40 phosphorylation, but strongly reduces AMPK phosphorylation.

FIG. 5 shows that Rapamycin blocks urolithin B-effect on the diameter of C2Cl2 myotubes.

FIG. 6 shows that Bicalutamide blocks urolithin B-effect on the diameter of C2Cl2 myotubes.

FIG. 7 shows that intravenous urolithin B prevents in mammal the weight loss of denervated muscle.

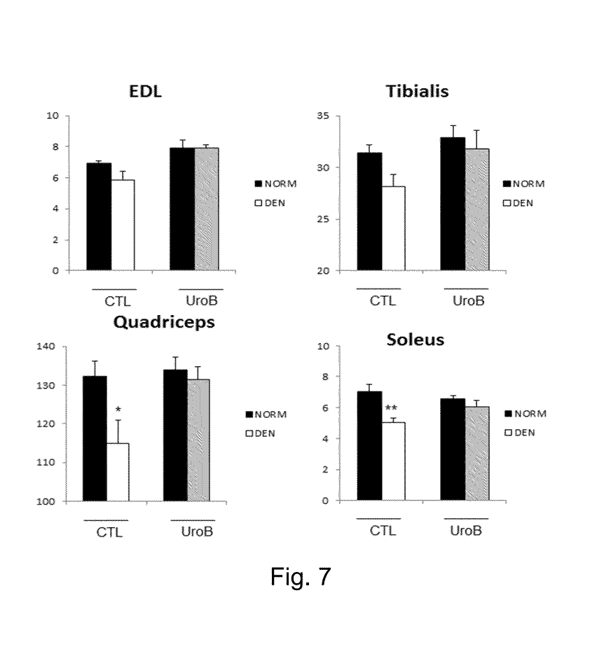

### DETAILED DESCRIPTION OF THE INVENTION

The inventors have searched for anti-inflammatory effect of several polyphenols on muscle cell anabolism, especially in the context of TNF-alpha-induced inflammation.

In this context, none of the tested compounds, except urolithin B, exhibited activity in significantly improving muscle cell growth and/or size.

However, urolithin B harbours weak antioxidant and/or anti-inflammatory properties.

Furthermore, the addition to muscle cells of urolithin B without the addition of TNF-alpha has resulted into improved muscle cell growth.

Therefore, these results can hardly be attributed to antioxidant and/or anti-inflammatory properties alleviating the TNF-alpha-induced stress.

The inventors then have elucidated the mode of action of urolithin B in enhancing muscle growth.

The inventors have found that urolithin B is an androgen-receptor agonist, but not the closely related urolithin A.

Therefore, urolithin B is a new molecule demonstrated as natural non-steroid androgen-receptor agonist.

This molecule can thus be used for the treatment or the prevention of osteoporosis or as ‘anabolisant’ (possibly in synergy with other compounds for muscle growth and/or repair). Indeed, Urolithin B administration (to a patient) results into increased muscle size, a fact compatible with a label as anabolisant, while the mechanism of action comprises reduction of muscle catabolism.

Furthermore, polyphenols mixtures being extracts containing precursors of urolithin B, such as pomegranate extracts and/or ellagitannins can hardly allow for a sufficient concentration of urolithin B in the blood, at least if not taken regularly and/or at a (very) high dosage: urolithin B is not the only ellagitannin-derived compound; urolithin B is rather a minor component; the blood concentration of urolithin is patient-dependent, since depending on the gut flora of the patient.

Therefore, a first aspect of the present invention is composition (for instance a food (and/or a beverage), feed or a pharmaceutical composition) comprising a sufficient amount of urolithin B, a derivative thereof, or a urolithin B precursor (and possibly a pharmaceutically or food or feed acceptable carrier).

A related aspect of the present invention is a food and/or a beverage or feed composition comprising a sufficient amount of urolithin B, a derivative thereof and/or a urolithin B precursor.

In the context of the present invention, urolithin B derivatives refers to glucosidic (e.g. glucuronic form), sulphated and alkylated forms. Preferably, these derivatives are the result of a covalent link on the (phenolic) —OH residue of urolithin B.

When urolithin B derivatives are expressed in weight units, this preferably refers to weight unit of urolithin B equivalents (i.e. the mole amount of urolithin B derivative multiplied by the molecular mass of non-derivatized urolithin; i.e. 212 g/mole).

Urolithin B (and derivative thereof) are preferred (over urolithin B precursors), for instance allowing injection or application through a dermal patch (such as over an injured muscle). In addition, even formulated for oral administration, urolithin B and derivative allow to apply (much) lower dose by comparison to urolithin B precursor, and the resulting blood content of urolithin B is independent of the patient's gut flora.

In the context of the present invention, “urolithin B precursor” refers to any edible extract comprising more than 15% (weight ellagitannins:dry weight) of ellagitannins and/or of ellagic acid (preferably more than 20%, still more preferably more than 25%, 30%, 35%, 40%, 50%, 75%; the percentages being weight ellagitannins and/or ellagic acid:dry weight extract).

Urolithin B precursor under the form of fruit (such as raspberry, strawberry, cloudberry, blackberry, pomegranate, grapes, nuts) extracts sufficiently enriched in ellagitannin (and/or ellagic acid) are encompassed in some aspects of the present invention, as well as other (non fruit) extracts sufficiently enriched in ellagitannin (and/or ellagic acid) and suitable for food consumption (such as some oak extracts being further refined).
These urolithin B precursors are encompassed by the present invention but, for instance, higher amounts of urolithin B precursor are needed to reach sufficient blood amount of urolithin B, since only a part will be converted. Furthermore, such precursors being much bigger molecules, larger amounts (in mass unit) are required, just to hydrolyse a given quantity of urolithin B.
When urolithin B precursors are expressed in ratios of weight units (e.g. the urolithin B precursor:leucine ratio), this preferably refers to weight unit of urolithin B equivalents (i.e. the mole amount of urolithin B precursor multiplied by the molecular mass of non-derivatized urolithin; i.e. 212 g/mole).

In the (food, feed and/or pharmaceutical) compositions of the present invention, urolithin B and urolithin B derivatives are advantageously present in an amount comprised between 0.05 ([weight urolithin B+weight urolithin B derivatives]:dry weight of the composition) and (almost) 100%, preferably between (about) 1% and (about) 50%, still more preferably between (about) 10% and (about) 25%.

In the (food, feed and/or pharmaceutical) compositions of the present invention, urolithin B, urolithin B derivatives and urolithin B precursors are advantageously present in an amount comprised between (about) 1% ([weight urolithin B+weight urolithin B derivatives+weight urolithin B precursors]:dry weight of the composition) and (almost) 100%, preferably between (about) 2% and (about) 50%, still more preferably between (about) 4% and (about) 40% and even between (about) 10% (or (about) 15%, (about) 20%, (about) 25%) and (about) 30%.

The (food, feed and/or pharmaceutical) compositions of the present invention can be a mixture of

(food, feed and/or pharmaceutical) compositions comprising urolithin B and urolithin B derivatives (such as between 0.05% ([w urolithin B+w urolithin B derivatives]:dry weight of the composition) and (almost) 100%, preferably between 1% and 50%, still more preferably between 10% and 25%) and of
(food, feed and/or pharmaceutical) compositions comprising urolithin B precursors (such as between 1 ([w urolithin B precursors]:dry weight of the composition) and (almost) 100%, preferably between 2% and 50%, still more preferably between 4% and 40% and even between 10% (or 15%, 20%, 25%) and 30%).

Advantageously, the (food, feed and/or pharmaceutical) compositions of the present invention (comprising urolithin B, derivatives and/or precursors) further comprise between (about) 1% (weight leucine:dry weight composition) and (about) 99%, preferably between (about) 5% and (about) 75%, more preferably between (about) 15% and (about) 50% of leucine.

Preferred (food, feed and/or pharmaceutical) compositions of the present invention comprise between (about) 0.05% (preferably 0.1%, more preferably 0.5%) and (about) 10% of urolithin B, and/or of urolithin B derivatives (weight[urolithin B+weight urolithin B derivative]:dry weight composition) and comprises between (about) 2% (weight leucine:dry weight composition) and (about) 99% of leucine, preferably between (about) 5% and (about) 90%, more preferably between (about) 25% and (about) 75%.

Other preferred (food, feed and/or pharmaceutical) compositions of the present invention comprise between (about) 10% and (about) 25% urolithin B, and/or of urolithin B derivatives (weight[urolithin B+weight urolithin B derivative]:dry weight composition) and comprises between (about) 2% (weight leucine:dry weight composition) and (about) 75% of leucine, preferably between (about) 5% and (about) 65%, more preferably between (about) 25% and (about) 60%.

Another possible food or feed composition of the present invention comprises between (about) 1% and (about) 75% of urolithin B precursor (weight urolithin B precursor:dry weight composition), and comprises between (about) 1% (weight leucine:dry weight composition) and (about) 99% of leucine, preferably between (about) 15% and (about) 75%, more preferably between (about) 25% and (about) 60%.

Another possible food or feed composition of the present invention comprise between (about) 25% and (about) 50% of urolithin B precursor (weight urolithin B precursor:dry weight composition), and comprises between (about) 2% (weight leucine:dry weight composition) and (about) 75% of leucine, preferably between (about) 15% and (about) 70%, more preferably between (about) 25% and (about) 60%.

Possibly, the leucine of food or feed compositions of the present invention is present in ingredients selected from the group consisting of plant extracts (such as soy hydrolysate, peas), (isolated) proteins, (partly)hydrolyzed proteins, branched amino acids (mixtures), isolated leucine and mixture thereof.

Thus possibly, further to the leucine, other amino acids including also taurine are present, especially in the food or feed compositions of the present invention, for instance in the form of added proteins, such as milk-proteins (whey), egg proteins, and/or in the form of added protein hydrolysates (e.g. whey hydrolysate, soy hydrolysate), possibly as full hydrolysate, and/or as isolated branched amino acids (e.g. mixtures consisting essentially of leucine, valine and isoleucine).

Alternatively, the amino acids of the (pharmaceutical) compositions of the present invention consists essentially of leucine.

Preferably, in the (food feed and/or pharmaceutical) compositions of the present invention (comprising urolithin B, derivatives and amino acids), the weight ratio of urolithin B (and/or urolithin B derivative) to leucine (possibly present as plant extract, as protein, as protein hydrolysates or among the branched amino acids) is comprised between (about) 1:1 and (about) 1:30, preferably between (about) 1:2 and (about) 1:15, more preferably between (about) 1:4 and (about) 1:8, wherein preferably the urolithin B derivative content is calculated as urolithin B equivalent upon the sum in moles of all of the urolithin B and urolithin B derivatives (possibly) present in these compositions, then upon the conversion (*212 g/mole) of the summed urolithin B moles to the urolithin B content in weight units (such as in grams).

Alternatively, in the (food feed and/or pharmaceutical) compositions of the present invention (comprising urolithin B precursors and amino acids), the weight ratio of urolithin B precursors to leucine (possibly present as plant extract, as protein, as protein hydrolysates or among the branched amino acids) is comprised between (about) 1:1 and (about) 1:30, preferably between (about) 1:2 and (about) 1:15, more preferably between (about) 1:4 and (about) 1:8, wherein preferably the urolithin B precursor content is calculated as urolithin B equivalent upon the sum in moles of all of the urolithin B precursors present in these compositions, then upon the conversion (mole number*212) of the summed moles (of urolithin B precursor) to the equivalent urolithin B content in weight units (such as in grams).

Advantageously, the food or feed compositions of the present invention comprising urolithin B (and derivatives and/or precursors and/or proteins such as whey or egg proteins and/or amino acids such as leucine) further comprise one or more additives selected from the group consisting of creatine, taurine, vitamins (preferably vitamin D), minerals (preferably Mg), isolated enzymes (preferably lactoferrin), polyphenols (ursolic acid, curcumin, resveratrol) and saccharides (such as glucose, fructose, sucrose and mixture thereof). For instance the compositions of the present invention comprise all of urolithin B (and derivatives and/or precursors and/or proteins and/or amino acids such as leucine), creatine and/or taurine, vitamins (preferably vitamin D), minerals (preferably Mg), and saccharides (such as glucose, fructose, sucrose), and possibly further comprises one or more of lactoferrin, ursolic acid, curcumin and resveratrol.

Advantageously, the food or feed compositions of the present invention comprising urolithin B (and derivatives and/or precursors and/or proteins such as leucine) further comprise between (about) 30 mg and (about) 10 g of creatine, preferably between (about) 100 mg and (about) 5 g, more preferably between (about) 300 mg and (about) 4 g more preferably between (about) 1 g and (about) 3 g.

Preferably, in the (food feed and/or pharmaceutical) compositions of the present invention (comprising urolithin B, derivatives and/or precursors), the weight ratio of urolithin B (and/or derivative and/or precursors thereof) to creatine is comprised between (about) 1:1 and (about) 1:30, preferably between (about) 1:2 and (about) 1:15, more preferably between (about) 1:4 and (about) 1:8, wherein preferably the urolithin B (precursor and/or derivative) content is calculated as urolithin B equivalent upon the sum in moles of all of the urolithin B, urolithin B derivatives and urolithin B precursors (possibly) present in these compositions, then upon the conversion (*212 g/mole) of the summed urolithin B moles to the urolithin B content in weight units (such as in grams).

Advantageously, these food, feed and/or pharmaceutical compositions comprising a sufficient amount of urolithin B (including precursors or derivative), and possibly comprising leucine and one or more additive, is taken on a daily basis (or several times (e.g. 2-4) per day or per week (e.g. 2-14) for at least 6 months (to allow for accumulation upon the time of urolithin B in the blood; for instance reaching a blood content (in a human being or in an animal selected from the group consisting of pigs, sheep, cattle, dogs and horse of at least 10 nM, or even of at least 500 nM, 1 μM, 3 μM, 5 μM, 10 μM and up to 15 μM of urolithin B (and/or urolithin B derivatives)).

Advantageously, these food, feed and/or pharmaceutical compositions comprising a sufficient amount of urolithin B (including precursors or derivative), and possibly comprising leucine and one or more additive, and possibly taken (by a human or by an animal selected from the group consisting of pigs, sheep, cattle, dogs and horse) on a daily basis for at least 6 months allows to reach blood level of urolithin B and/or of urolithin B derivative (independently of urolithin B precursor blood level, such as the blood level of urolithin A; this urolithin B precursor blood level being possibly (much) lower) comprised between (about) 10 nM and (about) 100 μM, preferably comprised between (about) 1 μM and (about) 50 μM, more preferably comprised between (about) 2.5 μM and (about) 15 μM.

Preferably, the sufficient amount of the urolithin B and/or urolithin B derivative present in the pharmaceutical composition of the present invention is comprised between (about) 0.5 mg and (about) 1 g, preferably between (about) 5 mg and (about) 200 mg, more preferably between (about) 50 mg and (about) 100 mg.

Preferably, the sufficient amount of the urolithin B precursor present in the pharmaceutical composition of the present invention is comprised between (about) 50 mg and (about) 5 g, preferably between (about) 100 mg and (about) 2 g, more preferably between (about) 200 mg and (about) 1 g.

Preferably, the sufficient amount of the urolithin B and/or urolithin B derivative present in the food or feed composition of the present invention is comprised between (about) 5 mg and (about) 2 Kgs, preferably between (about) 15 mg and (about) 50 g, more preferably between (about) 50 mg and (about) 10 g.

Preferably, the sufficient amount of the urolithin B precursor present in the food or feed composition of the present invention is comprised between (about) 5 mg and (about) 10 Kgs, preferably between (about) 200 mg and (about) 200 g, more preferably between (about) 500 mg and (about) 50 g.

Advantageously, this pharmaceutical composition comprising a sufficient amount of urolithin B precursor is formulated for oral administration.

Alternatively, this pharmaceutical composition comprising a sufficient amount of urolithin B or derivative is formulated for non-oral administration (e.g. by injection or in a dermal patch).

Another preferred aspect is a pharmaceutical composition comprising (i) a sufficient amount of urolithin B and/or of urolithin B derivative (i.e. at least 0.1% or at least 0.5% or even at least 1% ([w urolithin B+w urolithin B derivative]:dry weight composition; or at least 5 mg (or at least 10 mg or even at least 50 mg) of urolithin B and/or of urolithin B derivative), and (ii) from 0.01% (w:dry weight pharmaceutical composition) to (about) 20% (w:w) of a compound selected from the group consisting of capsaicin, menthol, trolamine salicylate, camphor, methylsalicylate, for instance this pharmaceutical composition is for topical administration (on an injured muscle), such as in the form of a dermal patch (where urolithin B synergizes with the compound (ii) in healing the injured muscle).

A related aspect of the present invention is urolithin B or a derivative thereof or the above described food, feed or pharmaceutical compositions according to the invention for use in enhancing muscle growth (in the elderity) or in preventing or treating muscle myopathy (in a mammal subject, preferably a human patient, for instance in a human having be diagnosed as having a genetic disposition for myopathy).

A closely related aspect of the present invention is urolithin B or a derivative thereof or the above described food, feed or pharmaceutical compositions according to the invention for use in the treatment or the prevention of osteoporosis in a mammal subject (preferably a human patient, such as a human patient diagnosed as suffering of osteoporosis or a human patient having a predisposition to osteoporosis).

Preferably, this pharmaceutical composition comprising urolithin B or a derivative thereof for use in enhancing muscle growth (in the elderity) or in preventing or treating muscle myopathy or for preventing or treating osteoporosis in a (human) patient further comprises leucine (possibly in the form of plant extract, of proteins, of protein hydrolysates, of mixture of amino acids or of isolated leucine).

Preferably, the urolithin B content of these compositions (for use in enhancing muscle growth (in the elderity) or in preventing or treating muscle myopathy or in preventing or treating osteoporosis in a (human) patient) is comprised between 0.1% (w_{urolithinB}:dry weight composition) and close to 100%, preferably between 0.5% and 75%, more preferably between 1% and 50%.

Preferably, the urolithin B precursor content of these composition (for use in enhancing muscle growth (in the elderity) or in preventing or treating muscle myopathy in a human patient) is comprised between 15% (w_{urolithinB precursor}:dry weight composition) and close to 100%, preferably between 20% and 75%, more preferably between 25% and 50%.

Still another related aspect of the present invention is urolithin B, a precursor or a derivative thereof and leucine (possibly in the form of plant extract, of proteins, of protein hydrolysates, of mixture of amino acids or of isolated leucine) or the above described food, feed or pharmaceutical compositions according to the invention for use in enhancing muscle growth and/or size (in the ageing) or in preventing or treating muscle myopathy in a mammal subject, preferably a human patient.

Preferably, leucine (added in the pharmaceutical composition of the present invention and/or in the pharmaceutical composition for use in restoring muscle function or in preventing or treating muscle myopathy) is isolated, and/or is exogenous to an extract comprising urolithin B and/or urolithin B derivative or urolithin B precursors.

Advantageously, the leucine of the pharmaceutical composition (and/or in the pharmaceutical composition for use in restoring muscle function or in preventing or treating muscle myopathy) is present in an amount between (about) 1 mg and (about) 10 g, such as about 6 g of leucine.

Preferably, this pharmaceutical composition (and/or the pharmaceutical composition for use in restoring muscle function or in preventing or treating muscle myopathy or in preventing or treating osteoporosis) further comprises creatine in an amount comprised between (about) 100 mg and (about) 10 g, more preferably between (about) 300 mg and (about) 5 g, still more preferably between (about) 1 g and (about) 3 g, and/or further comprises vitamins (preferably vitamin D), minerals (preferably Mg), and/or saccharides (such as glucose, fructose, sucrose), and possibly further comprises one or more of lactoferrin, ursolic acid, curcumin and resveratrol.

Still another related aspect of the present invention is a food or feed composition (an animal feed composition; preferably a feed composition for fish or for poultry or for mammals (horse, sheep, pigs, cattle, dogs)) comprising between (about) 5 mg and (about) 5 g of urolithin B (and/or of urolithin B derivative), preferably between (about) 50 mg and (about) 1 g, more preferably between (about) 200 mg and (about) 500 mg, and possibly between (about) 1 g and (about) 15 g of leucine (preferably between (about) 2 g and (about) 10 g of leucine).

Preferably, the urolithin B content of this food or feed composition is comprised between (about) 0.1% (weight urolithin B:dry weight composition) and close to 100%, preferably between (about) 0.5% and (about) 75%, more preferably between (about) 1% and (about) 50%, still more preferably between (about) 10% and (about) 25%.

Still another related aspect of the present invention is a food or feed composition (an animal feed composition; preferably a feed composition for fish or for poultry or for mammals (horse, sheep, pigs, cattle, dogs, camel)) comprising between (about) 50 mg and (about) 5 g of urolithin B precursor, preferably between (about) 200 mg and (about) 2 g, more preferably between (about) 500 mg and (about) 1 g, and possibly between (about) 1 g and (about) 15 g of leucine (preferably between (about) 2 g and (about) 10 g of leucine).

Preferably, the urolithin B precursor content of this (animal) feed composition is comprised between (about) 1% (weight_{urolithinB precursor}:dry weight composition) and close to 100%, preferably between (about) 20% and (about) 75%, more preferably between (about) 25% and (about) 50%.

Preferably, animals are selected from the group consisting of pigs, sheep, cattle, dogs, horse and (less preferably) camel, poultry or fish; hence the animal feed composition of the present invention is designed (quantitatively and qualitatively) for the selected animal, and is supplemented with urolithin B and/or urolithin B derivative and/or urolithin B precursor.

Still another related aspect of the present invention is an isolated non-steroid natural androgen-receptor agonist.

Preferably, this isolated non-steroid natural androgen-receptor agonist is urolithin B, a derivative or a precursor thereof.

Therefore another related aspect of the present invention is the (non therapeutical) use of urolithin B or a derivative thereof (or even of urolithin B precursor) for increasing muscle mass of a (non diseased) human being or of a (non-diseased) animal selected from the group consisting of pigs, sheep, cattle, dogs, horse and (less preferably) camel, poultry or fish.

Preferably, the urolithin B content and/or urolithin B derivative content (in a composition) for increasing muscle mass is comprised between 0.1% (w_{urolithinB}:dry weight composition) and close to 100%, preferably between 0.5% and 75%, more preferably between 1% and 50%.

Preferably, the urolithin B precursor content (in a composition) for increasing muscle mass is comprised between 15% (w_{urolithinB precursor}:dry weight composition) and close to 100%, preferably between 20% and 75%, more preferably between 25% and 50%.

### EXAMPLES

### Comparative Example

The inventors selected C2C12 murine skeletal muscle myoblasts for measuring the effect of polyphenols on muscle metabolism, especially when submitted to inflammatory stress (e.g. upon TNF-alpha exposure). In these conditions, anabolism is blocked.

Although rodents are not necessary suitable mammals (different pharmacokinetics of polyphenols derivatives than in human), isolated rodent cells can be used in the context of purified urolithin addition.

The inventors firstly selected urolithin A, as this metabolite was shown to have several advantageous properties; for instance, urolithin A is found in larger amount in blood of human beings than the other urolithins, and its anti-oxidant properties are bigger than those of urolithin B.

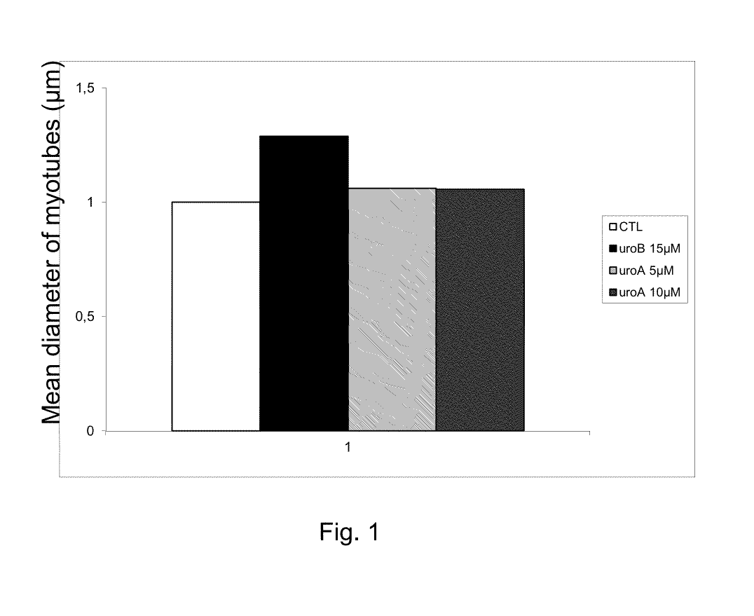

The addition of up to 10 μM of urolithin A does not increase the size of myotubes (see FIG. 1, conditions 3 and 4 in function of the control condition 1).

### Example 1

In order to further compare the anti-oxidant effect of urolithin towards muscle function, the inventors then selected urolithin B, which is the urolithin having the lowest antioxidant and anti-inflammatory properties.

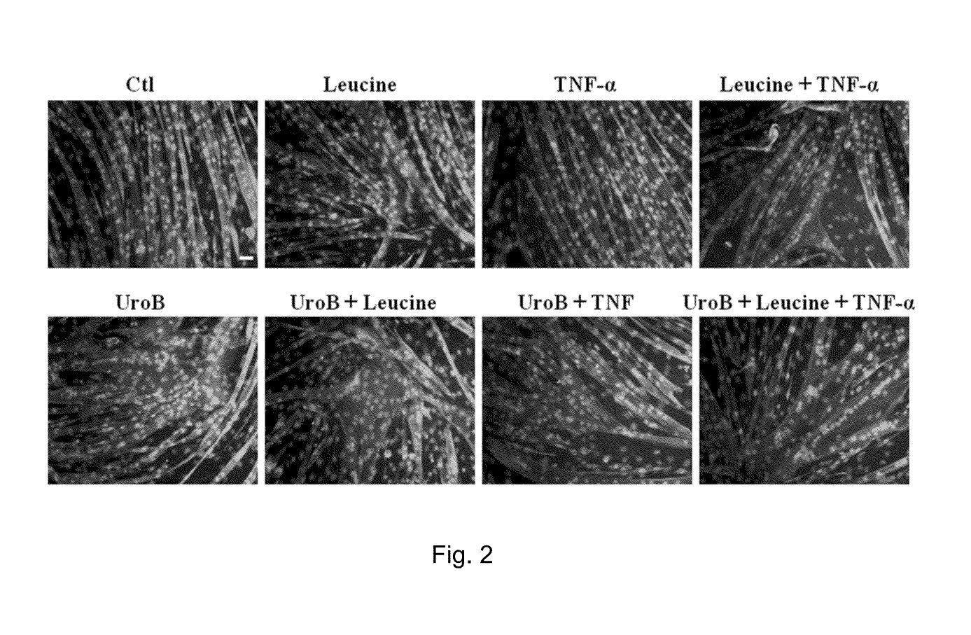

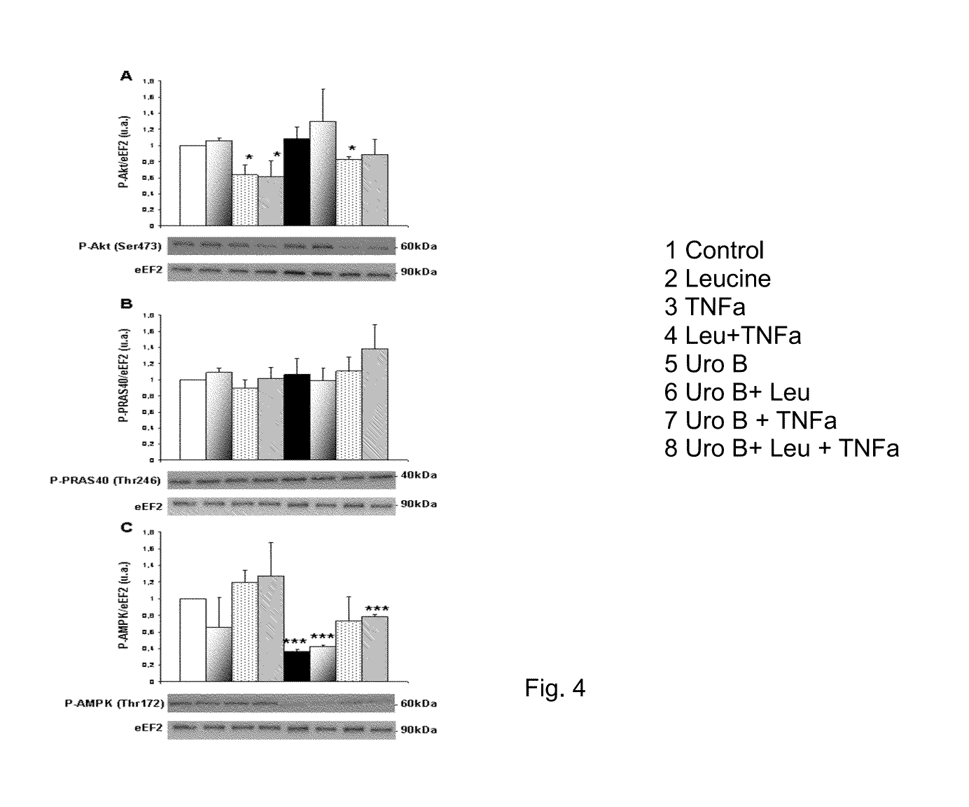

C2C12 myoblasts were differentiated for 4 days using 1% horse serum and 1% penicillin/streptomycin. The cells were thereafter grown for 24 h either with the same differentiation medium or with the differentiation medium supplemented with 15 μM urolithin B (conditions 5-8 of FIGS. 2-4 and of Table 1). Then the cells were grown either with the same differentiation medium or in the differentiation medium supplemented with 10 ng/mL TNF-alpha for 4 h (conditions 3-4 and 7-8 of FIGS. 2-4 and of Table 1). Thereafter, the cells were grown either with the same differentiation medium or in the differentiation medium supplemented with 5 mM leucine (conditions 2, 4, 6 and 8 of FIGS. 2-4 and of Table 1). The myotubes were labelled using anti-desmin antibodies, and nuclei labelling by Hoechst staining. Urolithin B consistently stimulates the diameter of myotubes.

The inventors have firstly measured if the addition of urolithin B presents some toxicity, but no toxicity was measured for concentrations of up to 40 μM.

The inventors have observed that urolithin B at 15 μM significantly increased the size of myotubes, and that this effect was resistant to the inflammatory stress (see Table 1, FIG. 1 and FIGS. 2A&B). Furthermore, under inflammatory stress, urolithin B and leucine are synergic, even at these high doses.

TABLE 1
mean diameter size of myotubes

| | |
|---|---|
| Ctl | 19.04 |
| Leucine | 25.5 |
| TNFa | 16.7 |
| Leucine + TNFa | 22.66 |
| uroB | 32.6 |
| uroB + Leucine | 28 |
| uroB + TNFa | 29 |
| uroB + TNFa + Leucine | 30.6 |

### Example 2

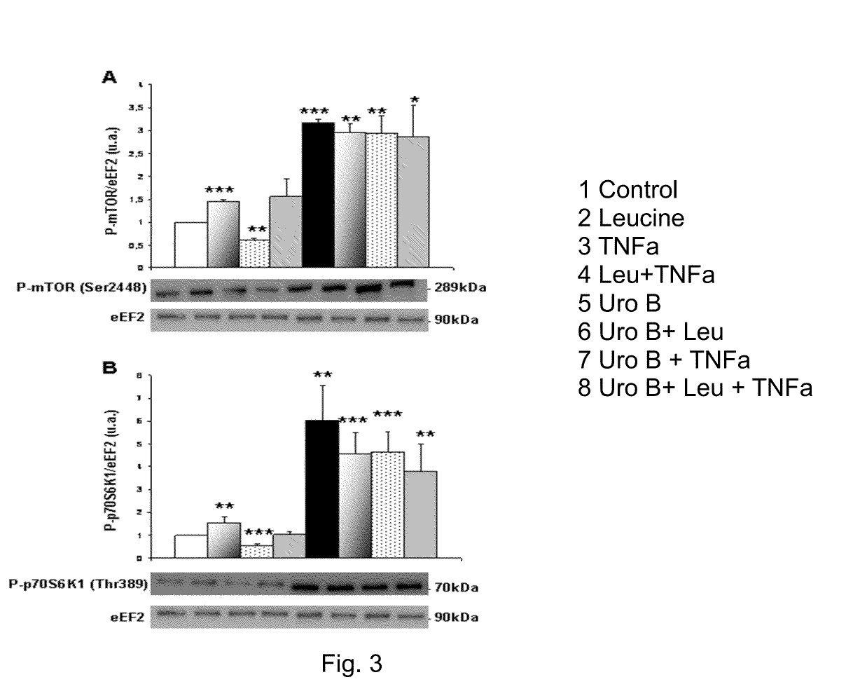

The inventors then refined the analysis in order to elucidate the underlining molecular pathway (FIGS. 3-6).

Among the pathways studied, the inventors have measured P70S6k phosphorylation to reflect protein synthesis and found a 5- to 6-fold induction upon urolithin B addition, but almost no induction upon urolithin A addition.

Surprisingly, co-addition of both urolithin A and B resulted into no induction of P70S6k phosphorylation. Therefore, such phenomenon is either due to toxicity of this specific composition or to an inhibitory effect of the urolithin B-induction by the presence of large amounts of urolithin A.

In other words, the inventors conclude that mixtures comprising urolithin B, urolithin B precursor(s) (and possibly other polyphenol derivatives) can only hardly have a positive effect on muscle anabolism. However mixtures comprising urolithin B, urolithin B precursor(s) (and other polyphenol derivatives) can be advantageously combined with amino acids extracts (e.g. leucine) in order to boost the urolithin B-driven effect. Indeed, although this effect is low, since the effective urolithin B dose is reduced in these conditions and/or the specific urolithin B-effect is antagonized by its precursors, the low effect can be synergically boosted by the addition of specific amino acids, such as leucine.

### Example 3

The inventors then have measured mTOR phosphorylation again to reflect protein synthesis and have observed a marginal leucine-induced phosphorylation and a strong urolithin B-induced phosphorylation.

The inventors then have measured Akt and PRAS40 phosphorylation still to analyse possible metabolism pathways. However, besides a tendency of synergy between the leucine and urolithin B addition, no major difference in phosphorylation patterns were observed.

On the other hand, the inventors have measured a strong reduction of AMPK phosphorylation upon urolithin B addition, again with a marginal bonus driven by leucine addition.

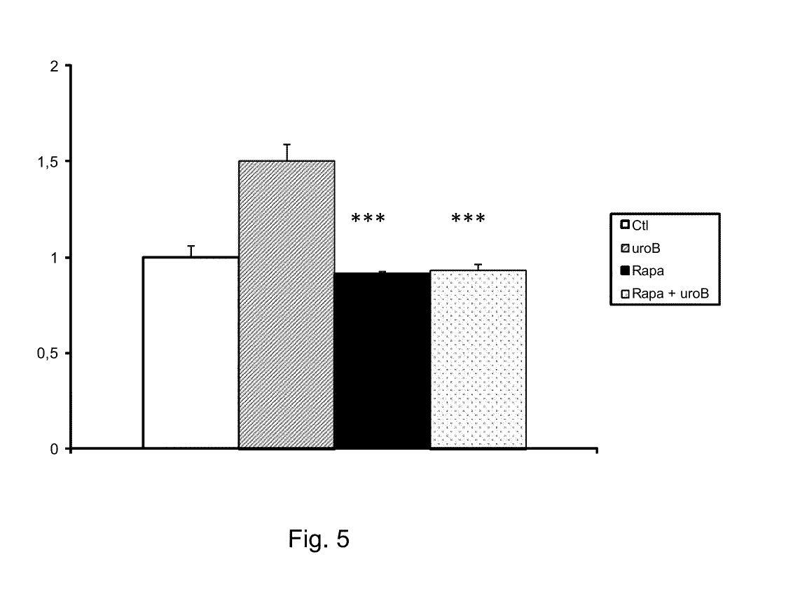

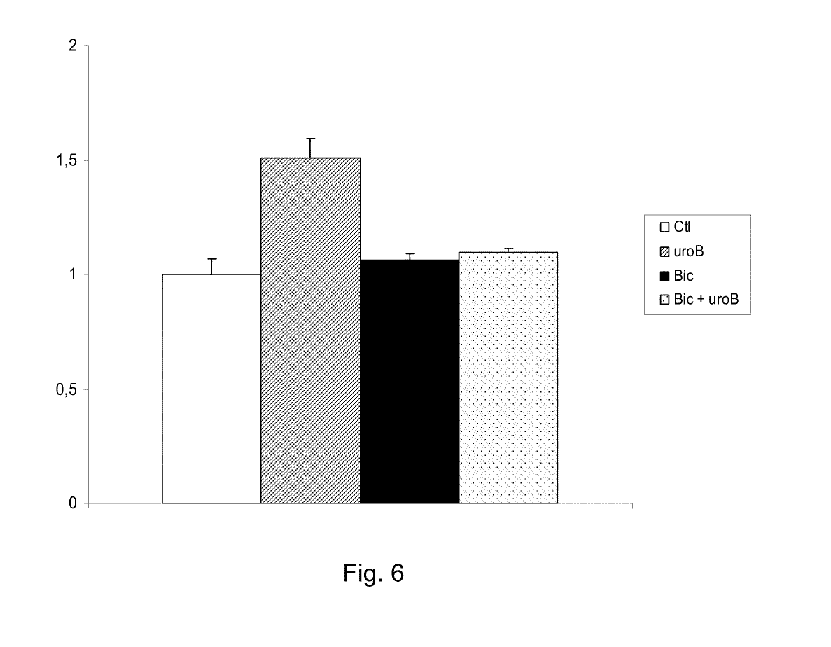

Finally, the inventors have measured the effect of rapamycin, on another mTOR inhibitor and of the anti-androgen bicalutamide (FIGS. 5-6 show the effect on the size of the muscular fibres) on the urolithin B-induced effects (including on the phosphorylation on mTOR, of AMPK, . . . ) and conclude that urolithin B acts upon binding and activating the androgen receptor, then by releasing the AMPK-inhibition on mTOR, increases protein synthesis in muscle cells, then the diameter of muscle fibres.

1.-26. (canceled)

27. A composition comprising:
at least 0.1% of an androgen receptor agonist being urolithin B and/or of urolithin B derivative (w[urolithin B+urolithin B derivative]:dry weight composition); or
at least 10% of a precursor of said non-steroidal androgen receptor agonist being urolithin B (w urolithin B precursor:dry weight composition); and
a sufficient amount of a compound of selected from the group consisting of testosterone, anti-myostatins, leucine and/or creatine.

28. The composition of claim 27, wherein the Urolithin B precursor is selected from the group consisting of ellagic acid or ellagitannins.

29. The composition of claim 27, wherein the anti-myostatin is selected from the group consisting of a soluble active Type II receptor, an anti-myostatin antibody or an anti-mysotatin siRNA.

30. The composition of claim 27 comprising at least 0.5% of urolithin B and/or of urolithin B derivative.

31. The composition of claim 27, wherein said urolithin B derivative is selected from the group consisting of glucosidic, sulphated and alkylated urolithin B forms.

32. The composition of claim 27, comprising at least 15% of urolithin B precursor.

33. The composition of claim 27, comprising at least 5% of leucine (w leucine:dry weight of the composition).

34. The composition of claim 27, comprising at least 100 mg of creatine.

35. The composition of claim 27, further comprising one or more additives selected from the group consisting of: vitamin D, magnesium, lactoferrin, ursolic acid, hydroxymethyl butyrate acid and mixtures thereof.

36. The composition of claim 27, further comprising at least 0.01% of a compound selected from the group consisting of: capsaicin, menthol, trolamine salicylate, camphor, methylsalicylate, hydroxymethyl butyrate (weight compound:dry weight pharmaceutical composition).

37. The composition of claim 27, comprising an edible composition.

38. The edible composition of claim 37, wherein the amount of urolithin B and/or of urolithin B precursor and/or of urolithin B derivative is sufficient for obtaining urolithin B and/or urolithin B derivative content of at least 100 nM in the blood of a mammal, wherein said urolithin B derivative is selected from the group consisting of glucosidic, sulphated and alkylated urolithin B forms.

39. The composition of claim 27, comprising a pharmaceutical composition with a further adequate pharmaceutical carrier

40. The pharmaceutical composition of claim 39, comprising a patch.

41. A method for enhancing muscle size in a mammal comprising administration of the composition of claim 27 to a mammal.

42. The method of claim 41 for reducing muscle weight loss resulting from muscle catabolism reduction.

43. The method of claim 41, for the prevention or treatment of myopathy in a mammal.

44. The method of claim 41, wherein the mammal is a human patient

45. The method of claim 43, wherein the mammal is a human patient having been diagnosed as having a genetic disposition for myopathy.

46. The method of claim 44, wherein the mammal is an elder human patient.
